(19) United States
(12) Patent Application Publication
(10) Pub. No.: US 2009/0043246 A1
(43) Pub. Date: Feb. 12, 2009

(54) Magnetic Surgical Device to Manipulate Tissue in Laparoscopic Surgeries Performed with a Single Trocar or Via Natural Orifices

(76) Inventor: Guillermo Manuel DOMINGUEZ, Buenos Aires (AR)

Correspondence Address: CROWELL & MORING LLP;INTELLECTUAL PROPERTY GROUP, P.O. BOX 14300, WASHINGTON, DC, 20044-4300, US

(21) Appl. No.: 11/834,746

(22) Filed: Aug. 7, 2007

(51) Int. Cl. A61N 1/18; A61B 18/14

(52) U.S. Cl. 604/21; 606/129; 606/144; 606/46

(57) ABSTRACT

The present invention is related to surgical instruments incorporating the usage of magnets for application in minimally invasive surgery to perform abdominal surgery with a single incision through the navel which is the most widely used, this incision can also be done through some natural orifice like the vagina, mouth, etc.

The present invention also describes a surgical tool to manipulate the magnetic surgical devices, a surgical apparatus to position an external magnet during surgery, a surgical probe comprising at least a magnet at one of its ends, a surgical tool to manipulate spherical magnets and washers, a cannula with a system to fasten knots and to fit a catheter and a organ surgical retractor.

Generally, the invention comprises instruments to perform cholecystectomy (gallbladder removal), but they are also useful for all type of operation requiring mobilization, traction, counter-traction or also separation of abdominal organs. They can be used in several kind of surgeries, such as laparoscopy, general, gynecological, urologic surgery, etc. surgery.

### FIELD OF THE INVENTION

The present invention is related to surgical instruments that incorporate the use of magnets for application in minimally invasive surgery to perform abdominal surgery with a single incision through the navel, this incision can also be done through some natural orifice like vagina, mouth or anus.

The present invention also describes surgical tools to manipulate the magnetic surgical devices, a surgical apparatus to position an external magnet during a surgery, a surgical probe that comprises at least one magnet in one of its ends, a surgical tool to manipulate spherical magnets, a catheter cannula with a system to fasten a preformed knot, and a surgical organ retractor.

Generally the invention comprises instruments to perform cholecystectomy (gallbladder removal), also used for all type of procedure requiring mobilization, traction, counter traction or also abdominal organs separation. They can be used in laparoscopic, gynecologic, urologic, pediatric surgeries.

### BACKGROUND OF THE INVENTION

In 1997 Dr. Fausto Davila Avila, in Mexico, invents a surgical technique which he calls “no track” surgery that features a single trocar usage, plus an optic with work canal aided by 1 to 1.2 mm diameter percutaneous needles and submit his paper to the Society of American Gastrointestinal Endoscopic Surgeons in 2004. This work is described in “Substitution of ports by percutaneous needles in endoscopic surgery” (“Sustitución de puertos por agujas percutáneas en cirugia endoscópica”). Rev. Mex. Cir. Endoscop. 2004; 5:172-178, Dávila F, Sandoval R, Montero Pérez J, Dávila Or, Dávila M, Alonso J, Lemus J.

Besides, the usage of magnets starts for several medical specialties. In Gatroeneterology: from December 2001 Villaverde A., Cope C at al. make compression gastroenteroanastomosis with magnets inserted via endoscopic or fluoroscopic means.

Related publications are Creation of compression gastroenterostomy by means of oral, percutaneous, or surgical introduction of magnets: feasibility study in swine. Cope C. JVIR 1995; 6:539-545. Evaluation of compression cholecystogastric and cholecystojejunal anastomoses in swine after peroral and surgical introduction of magnets. Cope C. JVIR 1995; 6:546-552. Stent placement of gastroenteric anastomoses formed by magnetic compression. Cope C; Clark T; Ginsberg G; Habecker P.JVIR 1999; 10:1379-1386. Long-term patency of experimental magnetic compression gastroenteric anastomoses achieved with covered stents. Cope C; Ginsberg G. Gastrointestinal Endoscopy June 2001 Volume 53 Number 7. Magnet usage for valves adjustments used in hydrocefalic patients. Neurocirugfa. 55(3): 627-630, September 2004. Jandial, Rahul M.D.; Aryan, Henry E.M.D.; Hughes, Samuel A.M.D., Ph.D. Collection, Michael L.M.D., Ph.D

Magnets are used for animals in robotic surgery. Dr. H. Rivas Magnetic positioning system to drive trocarles laparoscopic instruments. Dr Dimitri Oleynikov An in vivo mobile robot for surgical vision and task assistance, University of Nebraska.

U.S. Pat. No. 5,690,656 describes the creation of an anastomosis between two hollow viscera using magnets. Described are a method and a device to perform abdominal viscera anastomoses. To this end, a first magnet is set in place inside the first viscera, and a larger magnet inside the second viscera. The magnets attract each other so gripping a portion of the visceral walls between them. The method to perform an anastomosis of viscera comprises the following steps: (a) inserting a magnet in the viscera adjacent to a digestive zone; (b) inserting a second magnet in the stomach, thereby the second magnet is brought to the digestive zone until it attracts the first magnet and it is brought together in addition through a wall of the digestive zone and a visceral wall; and (c) Causing the fine tissue trapped between magnets experiences an ischemic necrosis.

The U.S. Patent Application 20030114731 describes to a method and apparatus to perform a surgery using a magnetic field. One or more magnets are used In the surgery arranged outside the patient body and they are used to position, to guide and to anchor surgical instruments and/or camera located inside the patient during surgery. The system is called MAGS (Magnetic anchoring and guidance system).

### SUMMARY OF THE INVENTION

The invention consists of using neodymium magnets, inside and outside the abdominal cavity, in different forms and sizes, biocompatible material, gold, silver, or nickel platted along with specially designed instruments to be used with magnets in laparoscopic surgery. The material going along with the magnets must be a nonferromagnetic steel or biocompatible medical grade plastic.

By means of magnets and the magnetic field generated through the abdominal wall is possible mobilize, push, pull, separate and elevate the organ or organs in order to perform 11 mm single incision in the navel surgery. Although the navel is the most frequently used site in laparoscopy, it is also possible to make this single incision with flexible surgical endoscopes entering the abdominal cavity by the vagina using optics with built-in work canal allowing instrument use. Taking advantage of the magnetic field generated by an external magnet and a magnet placed inside an abdominal cavity organ, plus the instruments entering the same orifice where the optics enters, different types of surgeries are performed.

Magnets are integral part of specially designed instruments to be used in laparoscopic or mini-invasive surgery. Preferably, the material going along with the magnets should be of a nonmagnetable steel or medical grade plastic. By means of these instruments, it is possible to mobilize, to pull, to separate or to elevate the organs to be able to perform the surgery making an about 11 mm single incision.

The device comprising the magnets enters the abdominal cavity by an umbilical trocar joints the tissue and is useful to mobilize organs, pull and push the organs. To this end, the magnets are gripped to the organ to be manipulated by means of clips, to be attracted latter and to be handled by external magnets. Also the magnets to be used latter can be inserted through the natural orifices like mouth, anus, vagina. They can also be set in place guided by radioscopy or endoscopy in the site of interest like the stomach, colon sigmoides, etc.

Also it is possible to push lithos from the biliary tract to the duodenum by type k108 probes with internal or spherical magnets of neodymium or ferromagnetic steel small balls set in place via transcystic or transcholedochus, and is progressed by external magnet for the movement within the biliary tract, this is guided by radioscopy. If the patient is female then a 12-15 mm trocar could be used, inserted via vagina (bottom of Douglas' sac) to perform the whole surgery through that orifice or for example, using mechanical sutures or optionally extraction of surgical pieces.

This technique with magnets also can be applied in traditional laparoscopic surgery, robotic surgery, surgery by natural orifices like accessory instruments of transgastrig, transvaginal surgery, etc.

These objects, will be apparent for the experts in the art by the following description.

### DESCRIPTION OF THE FIGURES

FIG. 1 shows a magnetic surgical device to manipulate tissue during a so called Dominguez tandem surgery according to a first object of the present invention.

FIG. 2 shows a layout of the clip for the anchoring of organs subject of the present invention.

FIG. 3 shows the organ anchoring clip fully assembled which is one of the objects of the present invention.

FIG. 4 shows in detail the annular magnet plurality threading which are integral part of the set in FIG. 1.

FIG. 5 shows a surgical tool called Thomas pliers used to manipulate a clip such as the one in FIGS. 2 and 3 according to a second object of the present invention.

FIG. 6 shows in detail the anchoring end of the so called Thomas pliers shown in FIG. 5.

FIG. 7 shows the so called Thomas pliers shown in FIG. 5 when used anchoring a clip from back.

FIG. 8 shows the so called Thomas pliers shown in FIG. 5 when used perpendicularly anchoring a clip.

FIG. 9 shows an external surgical magnet used to manipulate a magnetic surgical device like the one in FIG. 1, when this one is within the human body during surgery.

FIG. 10 shows a mechanical apparatus with a turn of up to 360° that is useful to manipulate an external surgical magnet like the one in FIG. 9 during surgery.

FIG. 11 shows probe comprising a plurality of magnets inside one of its ends, which is used in conjunction with a magnetic surgical device as shown in FIG. 1 to manipulate hollow organs.

FIG. 12 shows practical use of probes shown in FIG. 11.

FIG. 13 shows a surgical tool to manipulate spherical magnets or magnets with central orifice, called Camila pliers.

FIG. 14 shows a cannula with system to fasten knots called Valentina cannula which is used to take cholangiographies.

FIG. 15 shows in detail the end of the Valentina cannula as the one shown en FIG. 14, which is inserted into the patient.

FIG. 16 shows an organ separator or retractor called William separator or retractor.

### DESCRIPTION OF THE INVENTION

It is an object of the present invention to provide a magnetic surgical device to manipulate tissue during surgery comprising a grip which allows firmly hold tissue, the grip being made of a nonferromagnetic material; a magnetic system; and means of interconnection between the grip and said magnetic system, this means of interconnection being made of a nonferromagnetic material. In an embodiment, said grip can consist of a crocodile type clip which comprises two grips bind to each other, where said grips have a sawtooth profile on the side facing each other when the grip is closed, and where said grips have fins on the side they are not in contact with each other when the clip is closed; a pin that ties said grips and allows the joint among them; and a spring keeps these grips in contact in the closed position of the clip. Beside, this magnetic system can comprise an annular magnet plurality threading in a lead wire made of a nonferromagnetic material. Said annular magnets can be made of a rare earth material and be gold, nickel or silver plated.

It is another object of the present invention to provide a surgical tool to manipulate a crocodile type clip comprising a main body comprising hollow cylindrical tube; a first manipulation end, which comprises a handle with trigger which is kept separated from the handle by springs or iron straps; a second end for anchoring, which comprises a first grip and a second grip articulated between them; and an insert which runs within the main body and interconnects said first end with said second end so that when trigger is squeezed the grips in the end bind together. The tool is preferably made of a nonferromagnetic material. The second articulated anchoring element works together with the insert running inside the main body and with the trigger, so that this one turns as the trigger is squeezed. The first grip can include a recess at the end to receive a grip longitudinally during manipulation. The first grip can include a lateral recess to receive perpendicularly a grip during manipulation.

It is another object of the present invention to provide a surgical apparatus to position an external magnet during a surgery comprising a first arm coupled to a fixed point; a second arm, of adjustable length, rotatably connected to the first arm; a third arm, of adjustable length, rotatably connected to the second arm; and a grip rotatably connected to the third arm and which allows trapping an external magnet. In a preferred embodiment, the fixed point where the first arm couples to, can be at the surgery stretcher. The second arm can comprise orifices at both ends so that it is possible to vary the useful length of said second arm when varying the orifice in which this one is connected to the first arm and to the third arm. The third arm can comprise orifices at an end so that it is possible to vary the usable length of said third arm when varying the orifice in which this one is connected to the second arm. The surgical device can be driven in manual or robotic way. Also, if the invention is not available, the external magnet can be manually mobilized preferably the magnet being inside a gauntlet.

It is another object of the present invention to provide a surgical probe comprising at least a magnet in one of its ends. In the embodiment, the surgical probe is hollow and comprises at least a magnet inside one of its ends. The magnets can be made of rare earth material and be gold, nickel or silver plated. Also probes containing ferromagnetic steel small balls at an end can be used. Placed inside hollow organs, these small balls will be attracted and mobilized or anchored by magnets outside the hollow organ, allowing to move or to anchor the organ.

It is another object of the present invention to provide a surgical tool to manipulate spherical magnets or magnets with central orifice, comprising a main body comprising hollow cylindrical tube; a first manipulation end, which comprises a handle with driving device consisting of two arms articulated to each other so they open an close; a second anchoring end, which comprises at least three anchoring elements articulated to each other; and an insert running inside the main body and interconnecting said first end and said second end so that when the driving device is triggered when opening o closing the two arms articulated to each other, the anchoring elements at the anchoring end are joined or separated. The surgical tool is made of a nonferromagnetic material. The anchoring elements articulated to each other work together with the insert which runs inside of the main body and with the trigger.

It is another object of the present invention to provide a cannula, with a system to fasten preformed knots and for catheter comprising a main body made up by hollow cylindrical tube through which a catheter moves; a first manipulation end, which comprises a handle with an inlet orifice to the main body for the entrance of the catheter; a second operative end made up by hollow cylindrical tube, which comprises a lateral orifice whereby the thread comes out and a frontal orifice at the end whereby the catheter, which had entered by the first manipulation end, comes out, and whereby the thread with preformed external knot comes in. The lateral orifice of the second end can be partly beveled and partly comprises an edge so that in use, it allows to fasten a preformed knot. In order to fasten the knot it is necessary to push from the first manipulation end and pull in the opposite sense the thread running outside the cannula, all this through the work canal of the optics.

It is another object of the present invention to provide a surgical organ retractor comprising at least two grips which allow to firmly hold tissue, being these grips preferable made of a nonferromagnetic material; and a interconnection probe between said grips, said interconnection probe being made of a nonferromagnetic material. Said grips can consist of crocodile type clips comprising two grips ligated to each other, wherein said grips have a sawtooth profile on the side they are in contact to each other when the clip is closed, and wherein said grips have fins on the side they are not in contact to each other when the clip is closed; a bolt that ligates said grips and allows the articulation between them; and springs or straps which keep said grips in contact in the closed position of the clip.

It is another object of the present invention to provide a method of gallbladder extraction by means of laparoscopic surgery with the use of a single umbilical trocar aided by these devices, comprising the steps of making a pneumoperitoneum with carbon dioxide at usual pressure; to insert a trocar at umbilical level with closed or opened technique; to insert an optics with work canal through said trocar and to explore the abdominal cavity, then the optics is removed to insert the Dominguez tandem magnetic device through the trocar and then the optics is reinserted. In case of magnets and clip which latter will go through the optics canal, these optics movements could be avoided but probably magnetic field coercive force will be lost and the clip being smaller the ability to hold thicker tissues will be lost. After inserting a Dominguez tandem into the abdominal cavity through said trocar and by means Thomas pliers which enters by the optics operating canal, to see and to follow the positioning of the tandem that will be attracted by the magnetic field generated by an external magnet; the bottom of gallbladder is gripped with the tandem clip, aided in doing so by Thomas pliers, thus pulling the vesicular bottom towards cephalic over the liver and towards the patient right shoulder, this being achieved changing the position of the external magnet. To insert another Dominguez tandem through said trocar by means of a Thomas pliers; to position the Dominguez tandem using an external magnet leaving the crocodile type clip with its end directed towards the gallbladder; to take the gallbladder in infundibulum or Hartmann sac by opening of the crocodile type clip with Thomas pliers; to position the gallbladder properly by moving another external magnet which attracts by magnetic field the Dominguez tandem inside the patient; to move said Dominguez tandem towards the flank and towards the patient right iliac fossa to expose the Calot triangle that contains the cystic artery and the cystic conduit surrounded by peritoneum; to dissect the cystic conduit and the cystic artery with instruments which enter by the work canal; to repair the cystic conduit with external tie by means of a knot without fastening, one the ends of the thread coming out by the trocar; to make cysticotomy of the cystic conduit with scissors; to catheterize the cystic conduit by the work canal by using a Valentina cannula fastening the catheter through the orifice of Valentina cannula avoiding in this way the contrast fluid reflux and catheter displacement; the study is made, then the catheter is removed and the fastening of the knot is finished with Valentina cannula; optionally use clip. Clip or ligate the artery and section it; separate the gallbladder from the hepatic bed by dissection with instruments inserted by the optics canal for example the electro-scalpel and by moving the external magnets for the presentation of the gallbladder until the definitive separation of the gallbladder from the liver is achieved. Release the crocodile type clips with the Thomas pliers; take the cystic remaining that is left by the side of the gallbladder using the Thomas pliers; remove the Dominguez tandem once it is released from the magnetic field of the external magnet; to wash, to aspirate and to control for haemostasis; and to evacuate CO₂ by umbilical trocar and close. In addition, in the step of positioning the gallbladder by moving an external magnet, this can be made also by descending, instead of a Dominguez tandem, a magnet with a central orifice by means of a point in U with external-internal-external thread which is passed during its internal route, through the gallbladder and its two ends remain external to the umbilical trocar, then a magnet with orifice is threaded to one of the ends and a sliding knot is made (Gea or Roeder knot) behind the magnet in such a way that with a clamp to fasten knots, we slide the magnet towards the vesicular bottom.

In case of gallbladder under pressure, before any maneuver is previously evacuated by puncture and aspiration by the work canal. In case of thickened vesicular wall or escleraotrofic gallbladder, if the crocodile type clip cannot take the vesicular wall a magnet is inserted by means of U point transparietal to the gallbladder. Once the gallbladder is pulled with an external magnet, if adhesions exist they are treated with scissors or electro-scalpel. In the step of moving said Dominguez tandem towards the flank to expose the Calot triangle, if necessary, more Dominguez tandem are placed for greater tractive force and exhibition of the triangle. If the liver does not allow a easy dissection of the peritoneum and of the elements of the triangle, a William retractor is used or a needle with blunt end “string carrier” can be used, placed at right hypocondrium. In the step of catheterizing the cystic conduit, this can be made by introducing the catheter in percutaneous way, holding the same within the cystic with prehensile clamp by the canal. If string carrier needle is used it is possible to catheterize by this via and it is fastened within the cystic by prehensile clamp through the canal. In the step of removing the catheter and fastening the knot with Valentina cannula it is possible to place Hem-o-lok type clips to assure the closing of the conduit before its definitive section. If calculus in the biliary tract are found, use Dormina type baskets or are progressed to the duodenum by placing a spherical magnet in choledochus, and then by means of a capillary end containing a magnet, the espheric magnet is slid, dragging the calculum. In the step of removing the gallbladder taken by the remaining cystic, a sterile bag can be inserted and positioned with the crocodile type clip that was placed in infundibulum and a Thomas pliers by the canal, introducing then the gallbladder in bag and removing it so avoiding umbilical contamination.

It is another object of the present invention to provide a method to perform surgery of Hiatus comprising making a pneumoperitoneum with carbon dioxide at usual pressure; to insert to trocar at umbilical level; to insert an optics with work canal through said to trocar and to explore the abdominal cavity, to remove the optics to insert a Dominguez tandem by means of a Thomas pliers through said trocar and the optics so that the crocodile type clip will grip the stomach, said crocodile type clip being surrounded with a cover that avoids damage to the stomach; to position the Dominguez tandem by means of the usage of an external magnet remaining the crocodile type clip with its end directed towards the stomach; to insert a William retractor through said umbilical trocar by a Thomas pliers; to separate the left hepatic lobe by means of said William retractor to so expose the gastric esophagus union and the diaphragmatic pillars; to insert a surgical probe comprising at least one magnet at one of its ends through the mouth, positioning it in the stomach; to dissect and to close the pillars of the diaphragm by dissecting the esophagus and the esophagus-gastric union; to make a funduplicature to avoid the elevation of the stomach to the thorax and to avoid the hydrochlorate acid reflux from the stomach towards the esophagus by incompetence of the lower esophagic sphincter; to adjust the pillars and funduplicature by means of extracorporeal needles with thread and knots and/or continuous o separated sutures; to remove the surgical probe of the stomach; to remove Williams retractor; to remove the Dominguez tandem once it is released from the magnetic field of the external magnet; to wash, to aspirate and to control haemostasis; to evacuate CO₂ by the umbilical trocar and to close. In addition, if an hernia is detected, it is reduced. If diverticulm is detected, a magnet is placed inside the same by endoscopy and with an external magnet we mobilize it for its dissection outside of the esophagic wall in order to latter perform a resection on it and a to suture the esophagus. Optionally, in the fundiplicature the short vessels running from the stomach to the spleen are sectioned to allow a greater mobility of the stomach in the maneuvers before to the funduplicature, and a suture stomach-esophagus-stomach is performed so creating a valve that is calibrated with a surgical probe with magnets inserted by the mouth. It is furthermore possible to insert additional trocars by usual sites to use usual laparoscopy instruments.

It is another object of the present invention to provide a method to perform spleen surgery or splenectomy comprising: to perform a pneumoperitoneum with carbon dioxide at usual pressure; to insert trocar at umbilical level; to insert an optics with work canal through said to trocar; to insert a Dominguez tandem by means of a Thomas pliers through said trocar so that the crocodile type clip will grip the stomach, said crocodile type clip being surrounded at its toothed end with a cover that avoids damage to the stomach; to position the Dominguez tandem by means of the usage of an external magnet. To place another Dominguez tandem the crocodile type clip remaining with its end directed towards the spleen ligament to take the ligament aided by the Thomas pliers positioning with another external magnet until its traction is achieved; to insert a surgical probe comprising at least a magnet at one of its ends through the mouth positioning it in the stomach; to section all the ligaments connecting and anchoring the spleen, wherein the section is made on the ligament between spleen and tandem by cutting clamp entering by umbilical trocar; to release pedicle or splenius hilus whereby artery or arteries and vessel or vessels enter the spleen; to remove completely the released spleen by the navel placing it in a protective bag; to remove the surgical probe from the stomach; to remove the Dominguez tandem once it is released from the magnetic field of the external magnet; to wash, to aspirate and to control haemostasis; to evacuate CO₂ by the umbilical trocar and to close. It is possible to use a Williams' separator to separate left hepatic lobe in case this lobe does not allow the vision of spleen or of the spleen ligaments. It is possible to use another Williams' separator to hold the spleen and so achieve a better counter-traction thereof when the ligaments are released. It is possible to use one or more Dominguez tandem surrounded by a cover to manipulate, with the aid of an external magnet, the colon, intestines or stomach for better exhibition of the surgical field. In addition, it is possible to triturate the completely released spleen within a bag to remove it by the navel.

It is another object of the present invention to provide the usage of one or more magnetic surgical devices to manipulate tissue in Appendix and colon surgeries, a surgical tool to manipulate a crocodile type clip, one or more external magnets for handling mesoappendix-mesocolon, a surgical apparatus to position an external magnet during surgery, and a surgical probe comprising at least one magnet at one of its ends which is inserted through anus in the colon.

It is another object of the present invention to provide the usage of one or more magnetic surgical devices for gynecological surgeries placed by umbilical trocar and anchored to the uterus to manipulate the ovaries, the tubes, the uterus or infundibulum, a surgical tool to manipulate a crocodile type clip, one or more external magnets to drive the surgical magnetic devices, a surgical apparatus to position an external magnet during surgery, and an organ surgical retractor.

It is another object of the present invention to provide the usage, in abdominal wall surgeries, of one or more magnetic surgical devices for handling the peritoneum, a surgical tool to manipulate a type crocodile clip, one or more magnets to manipulate the surgical magnetic devices, and a surgical apparatus to position an external magnet during surgery.

It is another object of the present invention to provide the usage, in urologic surgeries, of one or more magnetic surgical devices for handling of the kidney, a surgical tool to manipulate a crocodile type clip, one or more external magnets to manipulate the surgical magnetic devices, a surgical apparatus to position an external magnet during surgery, a surgical probe comprising at least one magnet in one of its ends for placing in ureter by cystoscopy for location, identification and handling of the same, and a organ surgical retractor.

It is another object of the present invention to provide the usage, in achalasia surgeries, of one or more magnetic surgical devices, a surgical tool to manipulate a crocodile type clip, one or more external magnets to manipulate the surgical magnetic devices, a surgical apparatus to position an external magnet during surgery, a surgical probe comprising at least one magnet in one of its ends, and an organ surgical retractor.

It is another object of the present invention to provide the usage, in diverticles surgeries, of one or more magnetic surgical devices, a surgical tool to manipulate a crocodile type clip, one or more external magnets to manipulate the surgical magnetic devices, a surgical apparatus to position an external magnet during surgery, a surgical probe comprising at least one magnet in one of its ends, and an organ surgical retractor.

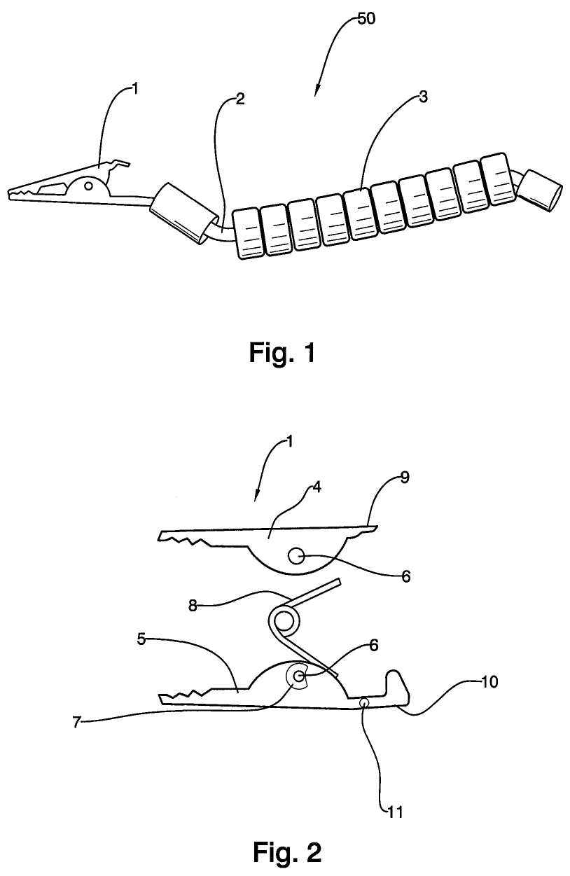

FIG. 1 shows a first embodiment of the present invention, which comprises a magnetic surgical device to manipulate tissue during a surgery 50 (herein called Dominguez tandem) made up by a crocodile type clip 1, which is coupled by means of a wire 2 with a magnet array 3. Clip 1 and all its components and wire 2 are preferably made of surgical steel or a nonferromagnetic biocompatible material.

Preferably, clips 1 should be smaller than 10 mm, of 10 to 50 mm in length. Preferably, the material can be Steel 304 SAE or 316 L or any other biocompatible material such as acetate of polyvinyl, titanium, etc.

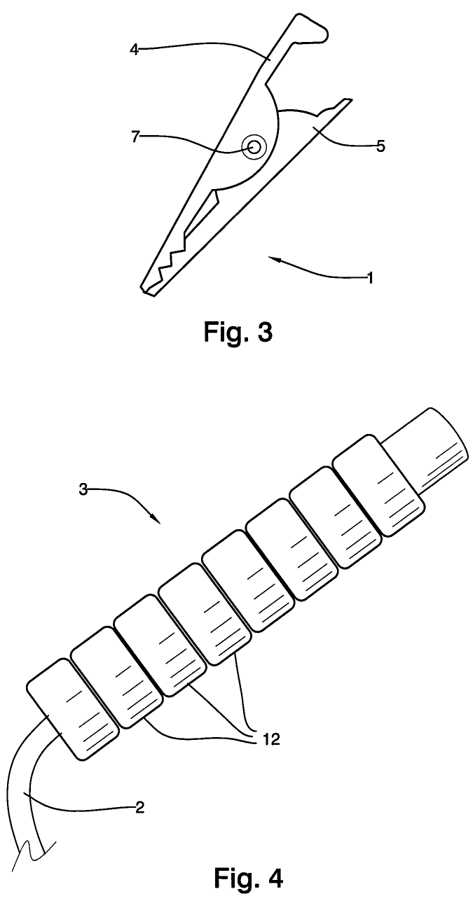

FIG. 2 shows a layout of the crocodile type clip 1. The clip comprises two grips 4, 5, which have in one of their ends, a sawtooth profile to facilitate gripping the human organ by the clamp. Both grips 4, 5 comprise a through orifice 6 by which they ligate by means of an anchoring bolt 7, which allows rotation movement between both grips 4, 5. Clip 1 comprises in addition a spring 8 that maintain the grips 4, 5 in closed position when no force is exerted on them.

In addition, grips 4, 5 comprise fins 9, 10 at opposite ends. These fins are designed to allow a force to be exerted on them to overcome the force of spring 8 and to cause the grips 4, 5 rotate on the anchoring bolt 7 coming away one from each other. Thus, the open clip is positioned on the human organ to be anchored, in order to latter release fins 9, 10 and allows the spring 8 close again grips 4, 5 on said organ. The grip 10 in addition comprises a through hole 11 by which a wire or no ferromagnetic thread is inserted.

FIG. 3 shows to the crocodile type clip of FIG. 2 in closed position. Here it is shown how the grips 4, 5 remain closed by the action of spring 8 (not shown).

FIG. 4 shows the magnet array 3 made up by a plurality of annular magnet 12. The magnets are built with any rare earth element and preferably with neodymium and it is possible to gold, nickel or silver plate the same. The magnets can have different shape and size, with or without orifice. Generally, it is preferable for them to be smaller than 10 mm to be introduced to the abdominal cavity by a trocar or by natural orifices. It is possible to insert them alone, guided by threads or probes or by means or pliers. The annular magnets must be threaded by a wire of a no ferromagnetic material.

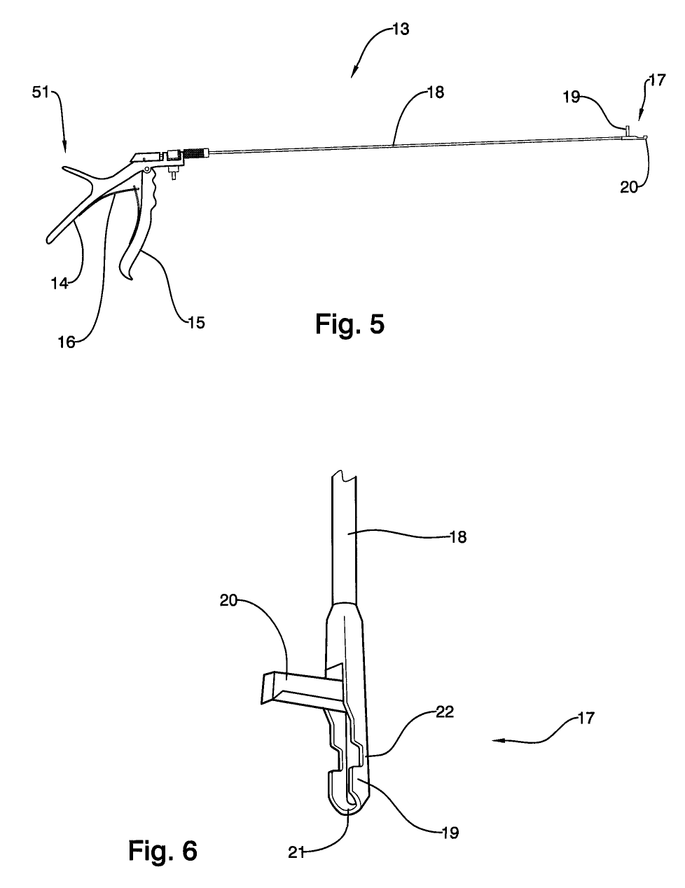

FIG. 5 shows pliers 13 (herein called Thomas pliers) designed to manipulate clips 1. The Thomas pliers 13 consists of a hollow cylindrical tube 18 through which runs an insert (not shown) that ligates both ends of the pliers. In one of its ends 51, the Thomas pliers 13 comprises a trigger 14 with a handle 15 which is kept apart form trigger 14 by means of springs or straps 16. At the opposite end 17, the Thomas pliers 13 comprises a pair of anchoring elements 19, 20 so that when the operator exerts pressure on handle 15 at the end 51 of the Thomas pliers, the anchoring elements 19, 20 at the other end 17 of the clamp are joined due to the displacement of the inner insert to remain en position of anchoring clip 1.

FIG. 6 shows the end 17 of the Thomas pliers 13 in detail. It is seen that the anchoring element 19 is fixed to and moves with the hollow main body 18. On the other side, the anchoring element 20 is articulated so that it is possible to be closed and to be opened with respect to the anchoring element 19 when through trigger 14, the insert that runs by the interior of hollow cylindrical tube 18 is driven. The profile of the anchoring elements 19, 20 is adapted to be able to manipulate clips 1. To this end, the anchoring element 19 comprises a recess 21 which allows to take clip 1 later on. In order to be able to anchored clip 1 perpendicularly, the anchoring end 19 comprises a side recess 22. The Thomas pliers 13 is able to anchor, to open, to laterally grip, to front grip, to back grip clips 13.

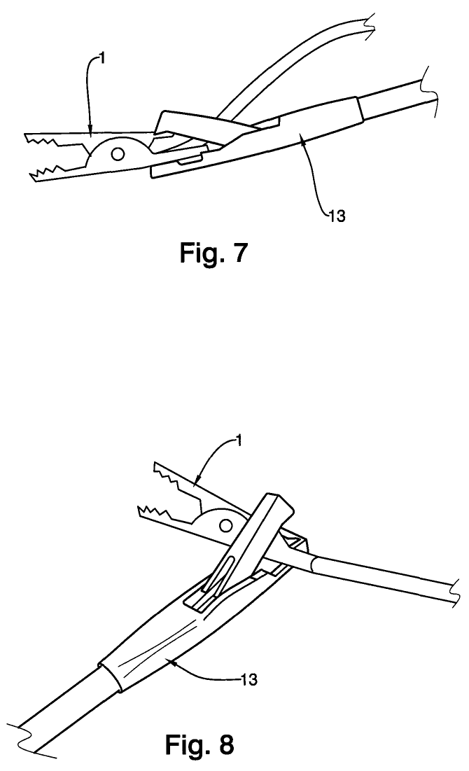

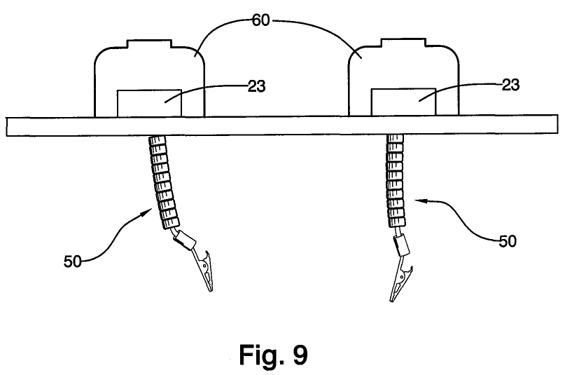

FIG. 7 shows how in practice Thomas pliers 13 holds clip 1 from the back. FIG. 8 shows in practice the anchoring of clip 1 perpendicularly. In use, the end of the Thomas pliers comprising trigger 14 and handle 15 remains outside the abdominal cavity, whereas the end 17 comprising anchoring elements 19, 20 is inserted in the abdominal cavity.

FIG. 9 shows a pair of gauntlets 60, each one of which houses an external magnet 23 that is used to manipulate a Dominguez tandem 50, which in use during surgery is within the human body. External magnets 23 can be permanent magnets or electromagnets; they should generate a magnetic field enough for the action required by the surgeon. The dimensions of magnet 23 will depend on the required use. In an preferred but not limiting embodiment, an external magnet can be 50×50×25 mm in size.

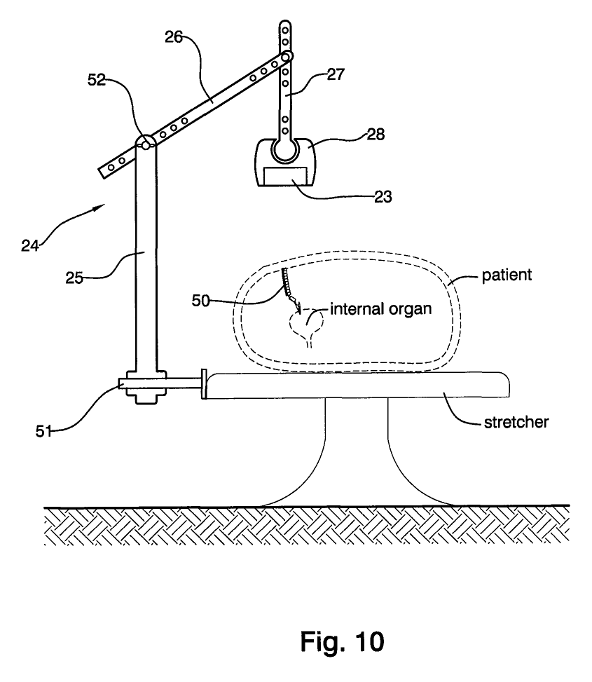

FIG. 10 shows a mechanical apparatus 24 with up 360° rotation, which is useful to manipulate external magnet 23 during surgery. In a preferred embodiment, the mechanical arm comprises a first arm 25 which can be coupled to the surgery stretcher or any other point designed to this end, a second arm with orifices 26 which allows to regulate the reach of the articulated set, and a third arm with orifices 27 that through a grip 28 anchors external magnet 23. First arm 25 is coupled to the operation stretcher or any other fixed point via a fastening device 51. At the other end, first arm 25 is coupled to the second arm 26 through a regulating device 52. This mechanical arm can be manual or robotic.

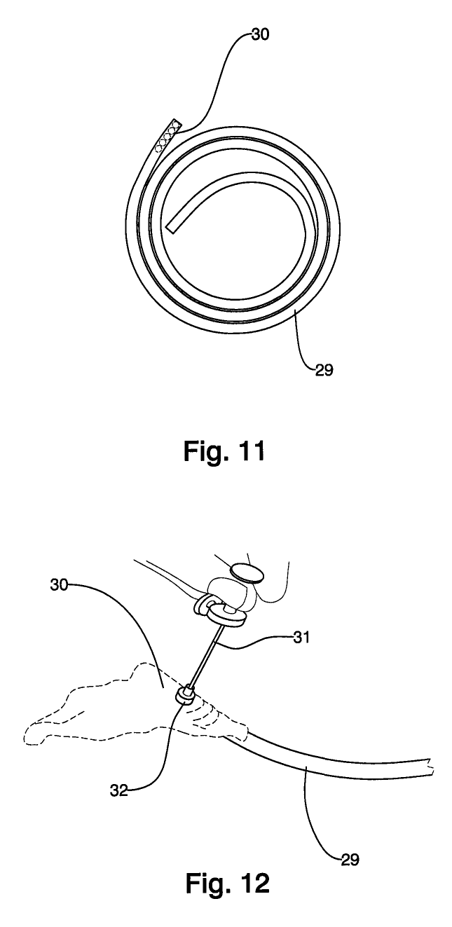

FIG. 11 shows a probe 29 comprising at one of its ends, a magnet plurality. This probe altogether is used together with the Dominguez tandem 50 to manipulate hollow organs like stomach, intestine, etc. In a first embodiment, probe 29 is hollow and comprises a magnet plurality 30 inside one of its ends.

In laparoscopic surgery, said probes are inserted in the human body through some natural orifice, preferably the mouth, the anus or the vagina. On the other hand, through the optics canal, Thomas pliers 13 is inserted holding the set made up by a crocodile type clip 1 and a magnet array 3. Then, probe 29 is arranged in magnetic contact by means of the magnetic attraction between magnets 30 of the probe with magnets 3 of system 50, so that the human tissue wall will be sandwiched between both magnet systems so being able to be properly manipulated, anchoring clip 1 of system 50, free, in the site selected by surgeon by anchoring all the set, aided by the Thomas pliers 13. Magnets 30, can be gold or nickel plated, can be different shapes, diameters, sizes, to be ingested with water or to be inserted by natural orifices with or without the use of endoscopy

FIG. 12 shows the use in practice, of the probe in FIG. 11. In the example, the probe is used in conjunction with a needle 31 comprising at its end a bell with a magnet 32. Needle 31 is inserted into the human body through the abdominal wall. Then, probe 29 and the magnet array 30 are arranged in magnetic contact with the needle 31 comprising at its end magnet 32, so that the human tissue wall remain sandwiched between both magnet arrays 30, 32, so being able to manipulate it properly. Needle 31 can be made of a steel 304 IS or 316L 1 mm in diameter. The bell containing magnet 32 and allowing the entrance of needle 31 can make of steel 304 IS or 316L.

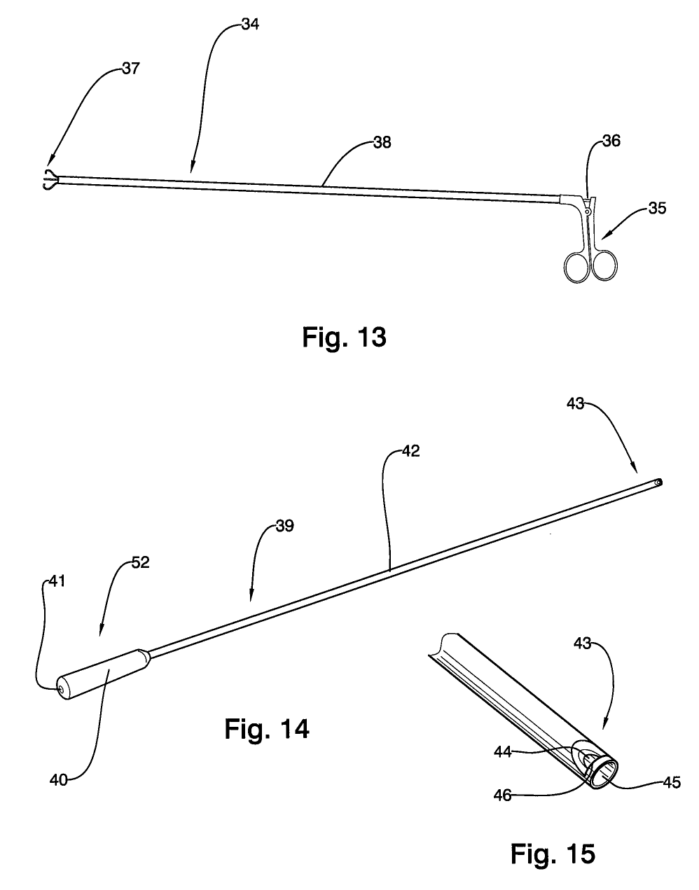

FIG. 13 shows pliers 34 (herein called Camila pliers) within the optics operating canal and the set within the trocar, which is used to manipulate spherical magnets or magnets with central orifice. Camila pliers 34 comprise a handle 35, a hollow cylinder 38 through which an insert 36 moves which engage handle 35 on the side and the other end is cut to originate 3 ends 37 that can be opened and be closed when handle 35 is driven, a hollow cylindrical tube 38 surrounds the solid cylinder or insert 36. The material used should be a no ferromagnetic surgical steel. As an illustration, but not in a limiting way, the dimensions of Camila pliers 34 can be 5 mm in diameter by 45 cm in length. Handle 35 can close or open the 3 ends 37 so holding spherical magnets or type washers magnets with internal orifice. These pliers are useful to place in or to remove from the abdominal cavity, spherical magnets and washers, or including the set of crocodile clamp 1 with magnets 3.

FIG. 14 shows a cannula 39 (herein called Valentina cannula) that is used to make cholangiografy (study of the biliary tract). The Valentina cannula 39 comprises a first end 52, a second operative end 43 and a hollow main body 42. The first end 52 comprises a handle 40 with an inlet orifice 41 for the entrance of a catheter.

FIG. 15 shows the second operative end 43 of a Valentina cannula 39 in detail, which is inserted into a patient. The end 43 comprises a side beveled orifice 44 and a frontal orifice 45. Side orifice 44 comprises an edge 46 that fasten a preformed knot. The catheter comes out and the thread enters by orifice 45 and the thread comes out by lateral orifice 44. The upper edge 46 serves to, when the catheter is within cystic conduit and preformed knot is outside the end, as the thread is pulled from outside and the Valentina cannula is pushed, the knot is fastened.

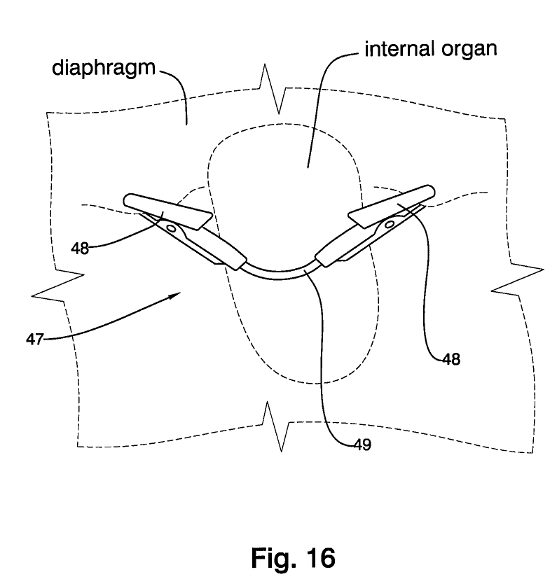

FIG. 16 shows a retractor or separator 47 (herein called William retractor or separator), which is used to retract organs during surgery to have thereby a better access to the organ that is being operated. Thus, in certain surgeries, to be able to see what is being operated, it is necessary to separate, for example, the liver, the uterus, the spleen, etc. William retractor 47 consists of two clips 48 jointed by a probe 49 in convenient length which works as hepatic, spleenic, uterine, etc -retractor. The organ lays on probe 49 and the clips 48 are set at several points example diaphragm-peritoneum to give tension and support in case of the left hepatic lobe, this maneuver allows to see the hiatus in case of hiatus hernia surgery, achalasia.

Several surgical techniques developed from previously described instruments are described bellow.

Cholecystectomy or Gallbladder Removal by Means of Laparoscopic Surgery with the Use of a Single Umbilical Trocar.

1-Introduction of trocar at umbilical level by means of closed or opened technique according to preference of the surgeon. Pneumoperitoneum with Veress' needle with carbon dioxide at usual pressure for closed technique. Optics with work canal is placed. Introduction of Dominguez tandem 50 (FIG. 1) which comprises magnets 3 in abdominal cavity by trocar. The Dominguez tandem can or cannot be taken by Thomas pliers 13 (FIG. 5) and tracked under direct vision by the optics.

2-Traction of the gallbladder bottom towards cephalic over the liver and towards patient right shoulder: Dominguez tandem 50 is inserted by trocar. Once in cavity Dominguez tandem 50 is positioned by means of use of an external magnet 23 (FIG. 9) remaining type crocodile clip 1 with its end directed towards the gallbladder. With Thomas pliers 13 the opening of the type crocodile clip 1 takes place and the gallbladder bottom is taken. By mobilizing the external magnet 23 which attracts by magnetic field the magnet array 3 inside the patient, the gallbladder is positioned as desire. This can also be achieved by lowering a magnet with an orifice by means of a thread that pass first through the organ and both ends remain outside, a magnet with orifice similar to a washer is threaded and a gea-roeder knot or any sliding knot running in a single sense fasten it with the tool to fasten knots to vesicular bottom.

In case of gallbladder under pressure, before any maneuver it can be previously evacuated by puncture and aspiration by the work canal, in case of thickened vesicular wall or escleraotrofic gallbladder, if the crocodile type clip 1 cannot take the vesicular wall, we lower a magnet as described above, by means of transparietal point to the gallbladder. Once the gallbladder is pulled by an external magnet 23, if adhesions exist they are treated in this step with scissors or electro-scalpel.

3-Exhibition of Calot triangle: Another Dominguez tandem 50 similar to the previous will take infundibulus or Hartmann sac and is mobilized with another external magnet 23, the assistant mobilizes the external magnet 23—Dominguez tandem 50—gallbladder towards flank and caudal to expose Calot triangle which contains the cystic artery and the cystic conduit surrounded by peritoneum. If necessary more Dominguez tandem 50 can be placed for stronger traction force and exhibition of the triangle. In this step if the liver does not allow an easy dissection of the peritoneum and the elements of the triangle, we can be helped with the William retractor 47 (sees FIG. 15) or with a string carrier needle (“needle of surgery without track”) placed at right hypochondrium. Then, to the dissection of cystic conduit and the cystic artery proceeds with instruments entering by work canal, being able to make ligatures with extracorporeal knots, place hem-o-lok clip or clip made of titanium LT200 bipolar energy or harmonic scalpel.

Cholangiography: Once identified the elements of the Calot triangle, the cystic conduit, it is repaired it with external ligature approaching a knot without fastening, the cysticotomy (eyelet in the conduit) with scissors is performed and it is catheterized by the work canal by using Valentina cannula 39 (see FIG. 14) fastening the catheter through orifice of Valentina cannula 39 avoiding in this way contrast fluid Triyoson® reflux and also catheter displacement is avoided. Another option is the introduction of the catheter in percutaneous way, holding it within the cystic with prehensile clamp by the canal, or also if string carrier needle were used it is possible to catheterize by this via and anchor it within the cystic by prehensile clamp by the canal. After performing the biliary tract test (cholangiography) the catheter is removed and knot is fastened with the Valentina cannula 39. If necessary, the Hem-o-lok type clip or clips are placed to assure the conduit closing before its definitive section. Artery is ligated o or a clip is placed to the artery and it is sectioned. In the case of finding calculus in the biliary tract, it is possible to try and make them to progress to duodenum by placing a spherical magnet in choledochum with the Camila pliers 34, and then by means of a bell with magnet 32 plus a needle 31, to make slide the spherical magnet, dragging the calculus.

4-Cholecystectomy: The gallbladder is detached from the liver. The necessary traction for the detachment of the gallbladder from the hepatic bed is achieved with external magnet 23 movements by the assistant. Coagulation and cut is performed as usual by electro-scalpel but from the work canal.

5-Extraction of Gallbladder: crocodile type clips 1 are released with Thomas pliers 13 at the same time the same Thomas pliers 13 takes the cystic remaining on the gallbladder side and it is removed under direct sight by trocar, once it is released from magnetic field of external magnet 23 Dominguez tandem 50 is removed. Another option according to the case and/or preference of the surgeon is to insert a sterile bag that is positioned with one of crocodile type clips 1 (the one of infundibulum) and Thomas pliers 13 by the canal, for the introduction of the gallbladder in the bag and extraction of the piece protected to avoid umbilical infection.

6-Wash aspiration and control of haemostasis, removal of Dominguez tandem 50, evacuation of CO₂ by umbilical trocar and close by planes this step in the usual way.

Surgeries of Hiatus for Pathologies like Achalasia, Hiatus Hernia and Esophagic Diverticulun

The surgery of hiatus and Achalasia are non-resective surgeries, that is to say, they do not remove organs, in the case of diverticulm is only resective for diverticulum.

For hiatus hernia treatment, which is a hernia, which generally takes place because of displacement of the stomach towards the thorax through the pillars of the diaphragm, we needed to separate the liver in order to expose these organs. We use, to this end, a William' retractor 47 to so separate the left hepatic lobe. The main objects of the surgery are to close the pillars that are the space through where the esophagus passes. What is normal is that the esophagus passes 1-2 cm from the thorax to the abdominal cavity through diaphragm between the pillars of the diaphragm. By closing the pillars, when hernia exists, the elevation of the stomach towards the thorax is avoided. After this step, a funduplicature is performed to avoid the elevation of the stomach to the thorax and to avoid cholhydric acid reflux from stomach towards the esophagus by incompetence of the lower esophagic sphincter, this is an involuntary muscle anatomically located at the esophagus-gastric union and is part of the wall of these organs and with normal pressure and function avoids the acid reflux. In order to close the pillars it is usually enough with the reduction of hernia and the settling of points between the pillars. The funduplicature is performed after dissection and close of pillars, the passage of gastric fundus 360 degrees on its axis behind the esophagus (Nissen operation with the stomach the esophagus is surrounded) etc.

If necessary, in this step, to give more mobility to the stomach the short vessels running from stomach to spleen are sectioned, with their due haemostatic control. Points stomach-esophagus-stomach is given so a valve is created, which is calibrated with a probe within the light of the esophagus-stomach that is introduced by the mouth.

With an hepatic Williams' retractor it is possible to expose the hiatus area and the dissection of the pillars of the diaphragm in usual way is begun, dissecting the esophagus and the esophagus-gastric union, in case of hernia, reduction of the same one, in case of Achalasia this step is not necessary and Heller miotomy is performed (a cut of 2 of the three layers of the esophagus-stomach, the serosa layer and muscular layer up to esophagic mucosa) according to technique with Electro scalpel.

In case of diverticulum, once it is individualized, a magnet within the light of the same by endoscopy is placed and with a Dominguez tandem we mobilize it for its dissection by outside the esophagic wall, once exposed diverticulum is resected and the esophagus is closed with sutures finishing the procedure with this step.

In this surgery probes with spherical magnets at the end are used, which enter by mouth to the stomach inserted by the anesthesiologist, it is further useful for calibrating the funduplicature and to help to mobilize the stomach during the procedure with a Dominguez tandem 50 which enters by umbilical trocar managing to move the esophagus and the stomach for the dissection of the pillars and hernia. With one or more Dominguez tandem and one external magnet organs are positioned and are pulled to carry out these steps with the aid of instruments that enter by the optics. In the case of Achalasia the technique is completed by suturing the stomach to the esophagic wall according to Dor technique (Heller-Dor' operation).

In difficult cases, usual laparoscopic instruments or 2-3 mm with more trocar can be used. Having ended the procedure the separator and the tandem are removed, CO₂ is evacuated and the umbilical wound is closed.

### Spleen Surgery (Splenectomy)

For splenectomy or spleen removal, spleen ligaments should be released. Once placed an umbilical trocar the section of the ligaments, which connect and anchor the spleen, is begun. This is achieved by placing a Dominguez tandem 50 in the ligament to be sectioned, it becomes tense with the aid of the external magnet, the section is performed on the ligament between spleen and the tandem by pliers which enters by umbilical trocar. In the same way proceeds with all ligaments. The William retractor-separator 47 is to separate the left hepatic lobe in case this lobe does not allow the sight of spleen or ligaments. The same can also be used for better counter-traction of spleen in releasing the ligaments (in this case the one which contacts on the separator probe is the spleen). If it is necessary to mobilize colon, intestine, stomach to gain better exhibition of the surgical field, this can be done with Dominguez tandem 50+external magnet (the clip which is used in these cases have no teeth or teeth of the clip are protected with a plastic that can be a clipping of a serum guide).

Release pedicle or splenic thread where artery or arteries and vessel or vessels enter the spleen is performed under haemostatic control with instruments inserted by the optics canal. After this step, the spleen is completely released; it must be finally removed, which can be performed by placing it in a protective bag as in the gallbladder case. The surgery uses a surgical probe containing spherical magnets at the end that enters by the stomach entrance inserted by the anesthetist, is useful to mobilize this latter with a Dominguez tandem 50 which enters by umbilical trocar for handling of splenic hilium. Finally, spleen is removed within the bag, by the navel. If necessary (because of size) it is morcelled within the bag and it is removed by navel. Tandem and retractor are removed, we control haemostasia, CO₂ is aspirated and navel is closed.

### Appendix and Colon Surgery

1-One or more Dominguez tandem 50 al used, plus an external magnet 23 for handling of mesoappendix-mesocolon or nontraumatic type clamp clip for tandem in wall of colon.

2-Probes of several diameters with magnets for colon which are inserted by the anus.

### Gynecological Surgery

1-One or more Dominguez tandem 50 for ovaries, tubes, uterus, infundibulum etc. plus an external magnet 23.

2-For mobilizing or anchoring the Uterus it is used William Retractor 47 or an intrauterine magnet (placed by hysteroscopy) with Dominguez tandem 50 placed by umbilical trocar which is anchored to the uterus. Both the intrauterine magnet and Dominguez tandem are handled with an external magnet 23.

### Surgery Abdominal Wall Surgery:

1-One or more Dominguez tandem 50 plus external magnets 23 are used for handling the peritoneum and Tap technique meshes (Transperineal) with trocar for inguinal and crural hernias. Idem for hernias.

2-Curved and straight steel needles with 150 cm long threads, measures 0.30-0.35-0.40 in diameter for extracorporeal knots and continuous or separated sutures.

### Urolologic Surgery

1-Spherical Magnet for uretral lithos.

2-One or more Dominguez tandem 50 are used, plus External magnet 23 for kidney.

3-William retractor 47 is used to separate the Liver.

4-Probes 23 with small magnet at the end for positioning inside urether by cystoscopy for location, identification and handling by an intra-abdominal magnet.

1. A magnetic surgical device to manipulate tissue during surgery comprising:
a grip that it allows to take hold of tissue firmly;
a magnetic system; and
means of interconnection between the grip and said magnetic system.

2. The magnetic surgical device of claim 1, wherein said grip and said means of interconnection are made of a nonferromagnetic material.

3. The magnetic surgical device of claim 1, wherein said grip consists of a crocodile type clip comprising:
two grips ligated to each other, said grips having sawtooth profiles on sides in contact to each other when the clip is closed, and said grips having fins on sides not in contact to each other when the clip is closed;
a bolt that ligates said grips and allows the articulation between them; and
a spring that keeps said grips in contact in the closed position of the clip.

4. The magnetic surgical device of claim 1, wherein said magnetic system comprises a plurality of annular magnets threaded in guide wire made of a nonferromagnetic material.

5. The magnetic surgical device of claim 3, wherein said annular magnets are made of a rare earth material and are gold, nickel or silver plated.

6. A surgical tool to manipulate a crocodile type clip comprising:
a main body made up by a hollow cylindrical tube;
a first manipulation end, which comprises a handle with trigger which is kept separated from the handle by a spring or an iron strap;
a second end for anchoring, which comprises a first anchoring element and a second anchoring element articulated to each other; and
an insert which runs within the main body and interconnects said first end with said second end so that when the trigger is squeezed at the manipulation end, the anchoring elements at the anchoring end bind together.

7. The surgical tool of claim 6, which is made of a nonferromagnetic material.

8. The surgical tool of claim 6, wherein the second articulated anchoring element works together with the insert running inside the main body and with the trigger so that it turns as the trigger is squeezed.

9. The surgical tool of claim 6, wherein the first anchoring element comprises a recess at the end to receive a grip longitudinally during manipulation

10. The surgical tool of claim 6, wherein the first anchoring element comprises a lateral recess to receive perpendicularly a grip during manipulation.

11. A surgical apparatus to position an external magnet during surgery comprising:
a first arm coupled to a fixed point;
a second arm, of adjustable length, rotatably connected to the first arm;
a third arm, of adjustable length, rotatably connected to the second arm; and
a grip rotatably connected to the third arm and which allows trapping an external magnet.

12. The surgical apparatus of claim 11, wherein the fixed point is at a surgery stretcher.

13. The surgical apparatus of claim 11, wherein the second arm comprises orifices in both ends so that it is possible to vary a usable length of said second arm when varying an orifice in which the second arm is connected to the first arm and the third arm.

14. The surgical apparatus of claim 11, wherein the third arm comprises orifices in an end so that it is possible to vary the usable length of said third arm when varying an orifice in which the third arm is connected to the second arm.

15. The surgical apparatus of claim 11, which can be driven in a manual or a robotic way.

16. A surgical probe comprising at least one magnet in one of its ends.

17. The surgical probe of claim 16, wherein said probe is hollow and has said magnet adhered inside one of its ends.

18. The surgical probe of claim 16, wherein the magnet is made of a rare earth material and is gold, nickel or silver plated.

19. A surgical tool to manipulate spherical magnets comprising:
a main body comprising a hollow cylindrical tube;
a first manipulation end, which comprises a handle with a driving device consisting of two arms articulated to each other so they open and close;
a second anchoring end, which comprises at least three anchoring elements articulated to each other; and
an insert running inside the main body and interconnecting said first end and said second end so that, when the driving device is triggered when opening or closing the two arms articulated to each other, the anchoring elements at the anchoring end are joined or separated.

20. The surgical tool of claim 19, which is made of a nonferromagnetic material.

21. The surgical tool of claim 19, wherein the anchoring elements articulated to each other work together with the insert which runs inside the main body and with a trigger.

22. A cannula with a system to fasten preformed knots comprising:
a main body made up by a hollow cylindrical tube through which a catheter and a thread move;
a first manipulation end, which comprises a handle with an inlet orifice to the main body for entrance of the catheter and the thread;
a second operative end made up by hollow cylindrical tube, which comprises a lateral orifice whereby the thread passes and a frontal orifice whereby the catheter, which had entered by the first end, passes, and whereby the thread with a preformed knot, which had also entered the first end, passes.

23. The cannula of claim 22, wherein the lateral orifice of the second end is partly beveled and partly comprises an edge so that in use it allows a preformed knot to be fastened.

24. A surgical organ retractor comprising:
at least two grips which allow to firmly hold tissue, these grips being made of a nonferromagnetic material; and
an interconnection probe between said grips, said interconnection probe being made of a nonferromagnetic material.

25. The magnetic surgical device of claim 24, wherein said grips and said interconnection probe are made of a nonferromagnetic material.

26. The surgical retractor of claim 24, wherein said grips consist of crocodile type clips which comprise:
two grips ligated to each other, said grips having sawtooth profiles on sides in contact to each other when the clip is closed, and said grips having fins on sides not in contact to each other when the clip is closed;
a bolt that ligates said grips and allows the articulation between them; and
a spring which keeps said grips in contact in the closed position of the clip.

27. A method of gallbladder extraction by way of laparoscopic surgery using a single umbilical trocar comprising the steps of:
performing a pneumoperitoneum with carbon dioxide at usual pressure;
inserting a trocar at umbilical level with closed or opened technique;
inserting optics with a work canal through said trocar to explore an abdominal cavity;
removing the optics to insert a tandem magnetic device through the trocar and then reinserting the optics;
inserting a tandem into the abdominal cavity through said trocar and by pliers, which enters by an optics operating canal;
seeing and following the positioning of the tandem at the same time it is attracted by an external magnetic field;
gripping the bottom of a gallbladder with a tandem clip of the tandem that is manipulated by the pliers,
pulling the vesicular bottom towards cephalic over the liver and towards the patient right shoulder by changing the position of an external magnet;
inserting another tandem through said trocar by way of pliers;
positioning the tandem using an external magnet, leaving a crocodile clip with its end directed towards the gallbladder;
taking the gallbladder in infundibulum or Hartmann sac by opening the crocodile clip with pliers;
positioning the gallbladder properly by moving another external magnet, which attracts, by magnetic fields, the tandem inside the patient;
moving said tandem towards a flank and towards the patient right iliac fossa to expose the Calot triangle that contains the cystic artery and the cystic conduit surrounded by the peritoneum;
dissecting the cystic conduit and the cystic artery with instruments which enter by the work canal;
repairing the cystic conduit with an external tie by way of a knot without fastening, one end of the thread coming out by the trocar;
making a cystictomy of the cystic conduit with scissors;
catheterizing the cystic conduit by the work canal using a cannula fastening the catheter through the orifice of the cannula, avoiding in this way contrast fluid reflux and catheter displacement;
removing the catheter and finishing the fastening of the knot with the cannula;
knotting and sectioning the artery;
separating the gallbladder from a hepatic bed by dissection with instruments inserted by the optics canal;
moving the external magnets for the presentation of the gallbladder until definitive separation of the gallbladder from the liver is achieved;
releasing the crocodile clips with the pliers;
taking cystic remaining that is left by the side of the gallbladder using the pliers;
removing the gallbladder under direct sight by umbilical trocar;
removing the tandem once it is released from the magnetic field of the external magnet;
washing, aspirating and controlling for haemostasis; and
evacuating CO₂ by umbilical trocar and closing.

28. The method of gallbladder extraction of claim 27, wherein the step of positioning the gallbladder is done by descending a magnet with a central orifice with an external-internal-external thread which is passed, during its internal route, through the gallbladder with its two ends remaining external to the umbilical trocar, threading a magnet with an orifice to one of the ends, and making a sliding knot behind the magnet in such a way that, with pliers to fasten knots, the magnet is slidable towards the vesicular bottom.

29. The method of gallbladder extraction of claim 27, wherein, when the gallbladder is under pressure, before any maneuver, it is previously evacuated by puncture and aspiration by the work canal.

30. The method of gallbladder extraction of claim 27, wherein, when a thickened vesicular wall or escleraotrofic gallbladder is present, if the crocodile clip cannot take the vesicular wall, a magnet is inserted by U point transparietal to the gallbladder.

31. The method of gallbladder extraction of claim 27, wherein once the gallbladder is pulled with an external magnet, if adhesions exist they are treated with scissors or electro-scalpel.

32. The method of gallbladder extraction of claim 27, wherein when moving said tandem towards the flank to expose the Calot triangle, if necessary, an additional tandem is placed for greater tractive force and exhibition of the triangle.

33. The method of gallbladder extraction of claim 27, wherein if the liver does not allow easy dissection of the peritoneum and of the elements of the triangle, a retractor or a needle with a blunt end “string carrier” is placed at right hypocondrium.

34. The method of gallbladder extraction of claim 27, wherein the step of catheterizing the cystic conduit is done by introducing the catheter in percutaneous way, and further comprising holding the same within the cystic with a prehensile clamp by the canal.

35. The method of gallbladder extraction of claim 27, wherein when a string carrier needle is used, it is possible to catheterize by way of the string carrier needle, which is fastened within the cystic by a prehensile clamp through the canal.

36. The method of gallbladder extraction of claim 27, wherein when removing the catheter and fastening the knot, it is possible to place clips to assure closing of the conduit before its definitive section.

37. The method of gallbladder extraction of claim 27, wherein when calculum in the biliary tract are found, baskets are used or progressed to the duodenum by placing a spherical magnet in choledochus, and then, by way of a capillary end containing a magnet, the spherical magnet is slid, dragging the calculum.

38. The method of gallbladder extraction of claim 27, wherein when removing the gallbladder, a sterile bag can be inserted and positioned with the crocodile clip that was placed in infundibulum and a pliers by the canal, and the gallbladder is then introduced into the bag and removed, so avoiding umbilical contamination.

39. A method to perform surgery, comprising:
making a pneumoperitoneum with carbon dioxide at usual pressure;
inserting a trocar at umbilical level;
inserting optics with a work canal through said trocar to explore the abdominal cavity;
removing the optics to insert a tandem by means of pliers through said trocar and said optics so that the crocodile clip will grip the stomach, said crocodile clip being surrounded with a cover that avoids damage to the stomach;
positioning the tandem using an external magnet with the crocodile type clip remaining with its end directed towards the stomach;
inserting a retractor through said umbilical trocar with pliers;
separating the left hepatic lobe using said retractor to expose the gastric esophagus union and the diaphragmatic pillars;
inserting a surgical probe comprising at least one magnet at one of its ends through the mouth, and positioning it in the stomach;
dissecting to close the pillars of the diaphragm by dissecting the esophagus and the esophagus-gastric union;
making a funduplicature to avoid the elevation of the stomach to the thorax and to avoid hydrochlorate acid reflux from the stomach towards the esophagus by incompetence of the lower esophagic sphincter;
adjusting the pillars and funduplicature by extracorporeal needles with thread and knots and/or continuous or separated sutures;
removing the surgical probe of the stomach;
removing the retractor;
removing the tandem once it is released from the magnetic field of the external magnet;
washing, aspirating and controlling for haemostasis; and
evacuating CO₂ by the umbilical trocar and closing.

40. The method to perform surgery of claim 39, wherein if a hernia is detected, it is reduced.

41. The method to perform surgery of claim 39, wherein if diverticulm is detected, a magnet is placed inside the same by endoscopy and, with an external magnet, is mobilized for dissection outside of the esophagic wall in order to later perform a resection on it.

42. The method to perform surgery of claim 39, wherein, in the fundiplicature, the short vessels running from the stomach to the spleen are sectioned to allow a greater mobility of the stomach, and a stomach-esophagus-stomach suture is performed to create a valve that is calibrated with a surgical probe inserted by the mouth.

43. The method to perform surgery of claim 39, wherein additional trocars are inserted by usual sites to use usual laparoscopy instruments.

44. A method to perform spleen surgery or splenectomy comprising:
performing a pneumoperitoneum with carbon dioxide at usual pressure;
inserting a trocar at umbilical level;
inserting optics with a work canal through said trocar;
inserting a tandem by way of pliers through said trocar so that a crocodile clip will grip the stomach, said crocodile clip being surrounded at its toothed end with a cover that avoids damage to the stomach;
positioning the tandem using an external magnet;
placing another tandem, the crocodile type clip remaining with its end directed towards the spleen ligament to take the ligament aided by the pliers with another external magnet until traction is achieved;
inserting a surgical probe, comprising at least a magnet at one of its ends, through the mouth, and positioning it in the stomach;
sectioning all ligaments connecting and anchoring the spleen, wherein the section is made on the ligament between the spleen and the tandem by a cutting clamp entering by umbilical trocar;
releasing pedicle or splenius hilus, whereby artery or arteries and vessel or vessels enter the spleen;
removing completely the released spleen by way of the navel, and placing it in a protective bag;
removing the surgical probe from the stomach;
removing the tandem once it is released from the magnetic field of the external magnet;
washing, aspirating and controlling for haemostasis; and
evacuating CO₂ by the umbilical trocar and closing.

45. The method to perform spleen surgery of claim 44, wherein a separator is used to separate the left hepatic lobe when this lobe does not allow the vision of spleen or of the spleen ligaments.

46. The method to perform spleen surgery of claim 44, wherein a separator is used to hold the spleen and so achieve a better counter-traction thereof when the ligaments are released.

47. The method to perform spleen surgery of claim 44, wherein at least one tandem surrounded by a cover is used to manipulate, with the aid of an external magnet, the colon, intestines or stomach for better exhibition of the surgical field.

48. The method to perform spleen surgery of claim 44, wherein a completely released spleen is triturated within a bag to remove it by the navel.
